(19) United States
(12) Patent Application Publication
(10) Pub. No.: US 2016/0161579 A1
(43) Pub. Date: Jun. 9, 2016

(54) COMBINED MRI PET IMAGING

(71) Applicant: KONINKLIJKE PHILIPS N.V., Eindhoven (NL)

(72) Inventor: Johan Samuel VAN DEN BRINK, Meteren (NL)

(21) Appl. No.: 14/906,945

(22) PCT Filed: Jul. 23, 2014
(86) PCT No.: PCT/EP2014/065768

§ 371 Date: Jan. 22, 2016

(30) Foreign Application Priority Data
Jul. 30, 2013 (EP) 13178496.9

(51) Int. Cl. G01R 33/48; G01T 1/16; A61B 5/00

(52) U.S. Cl. CPC G01R 33/481; A61B 5/4848; G01T 1/1603

(57) ABSTRACT

Combined use is made of image values at corresponding image locations defined by amide proton transfer MRI image data and 18F-FLT, 11C-MET, or 18F-FDG PET image data. The combined use may include computing multimodal heterogeneity for combined PET and amide proton transfer MRI image values, using PET image data to distinguish different image locations during processing and/or display of amide proton transfer image data, and tissue classification based on combinations of values derived from the amide proton transfer MRI and/or PET images.

### TECHNICAL FIELD

The invention relates to the field of PET-MRI. The invention also relates to a computer program for MRI imaging and to an MRI imaging system and method. The invention particularly relates to MRI imaging for the purpose of assessing treatment response, notably for use in treatment schemes for oncology and stroke, and in drug discovery for medicines applicable in these treatments.

### BACKGROUND OF THE INVENTION

A form of multimodal magnetic resonance imaging (MRI) and positron emission tomography (PET) is described by Laymon et al. in an article titled “Combined imaging biomarkers for therapy evaluation in glioblastoma multiforme: correlating sodium MRI and F-18 FLT PET on a voxel-wise basis”, published in Magnetic Resonance Imaging. 2012 November;30(9):1268-78.

Laymon et al. study assessment of response to cancer therapy. Laymon et al use Na MRI images and PET images obtained with 18F-FLT (fluorothymidine with the 18F isotope of fluor). Laymon et al report that the two modalities may provide complementary information regarding tumor progression and response. In addition Laymon uses a 3T structural MRI scan as a baseline to register the Na MRI and PET images.

Conventionally, 18F-FLT or another PET tracer such as 18F-FDG (fluorodeoxyglucose with the 18F isotope) is used for oncologic treatment planning In proliferating cells, upregulated DNA synthesis requires increased amounts of thymidine, which shows up 18F-FLT in certain cell types. In addition, 18F FLT imaging shows intra-tumour heterogeneity of proliferation in certain tumours to correlate with predicted chemotherapeutic response (J Nucl Med 2012; 53 (Supplement 1):387).

FDG is a glucose that accumulates in cells. Although its version with 18F is usually detected by means of PET, Rivlin et al. have suggested that FDG or the non-fluorinated compound 2DG (deoxyglucose) can also be detected by means of Chemical Exchange-dependent Saturation Transfer MRI (CEST-MRI). See “Chemical exchange saturation transfer (CEST) MRI of 2DG and FDG as a tool for molecular imaging of tumors and metastases” in Proc. Intl. Soc. Mag. Reson. Med. 21 (2013) page 425.

However, the required in-vivo tracer concentrations for detection of DG with CEST MRI may reach toxic levels. PET scans are burdensome because they involve radioactivity. 18F-FLT and 18-FDG used in such scans may expose patients to radiation toxicity. Therefore it is desirable to administer only limited quantities.

Further, the US-patent application US2009/0324035 discloses a method to combine multiple binary cluster maps. Each cluster map represents characteristic information, eg, from MR-BOLD, PET or CEST MR data. Each cluster is assigned a reliability factor. Information of the binary cluster maps is combined into a single cluster map and a reliabilty factor is assigned to the single cluster map.

### SUMMARY OF THE INVENTION

Among others it is an object to provide a method for improved detection, localization and characterization of cell proliferation with PET- MRI.

A computer program product with instructions for a programmable image processing system is provided that, when executed by the programmable image processing system, will make the programmable image processing system perform the steps of
obtaining amide proton transfer MRI image data;
obtaining 18F-FLT, 11C-MET, or 18F-FDG PET image data;
making combined use of image values at corresponding image locations defined by the amide proton transfer MRI image data and the PET image data. The computer program product may comprise a machine readable medium containing the instructions for the image processing system, such as an optical or magnetic disk, or a semi-conductor memory, e.g. a non-volatile semi-conductor memory. To make combined use of the amide proton transfer MRI image data and 18F-FLT, 11C-MET, or 18F-FDG PET image data, amide proton transfer MRI and 18F-FLT, 11C-MET, or 18F-FDG PET images may be registered, i.e. a map may be determined that map locations in the image spaces onto each other that represent the same location in a subject.

Preferably, the combined use of image values of the amide proton transfer (APT)-MRI image data and the PET image data is carried-out to reconstruct a multi-modal image of which the image data are combined in that the image values of the multi-modal image depend on both the APT-MRI image data and the PET image data. This multi-modal image may have image values such that the colour rendition or the contrast rendition is dependent on image values of each of the APT-MRI image data and the PET image data. For example the colour or contrast rendition of e.g. the APT-MRI image is adapted on the basis of the image values of the PET-image. In another implementation a local heterogeneity estimate is made on the basis of the image values of both the APT-MRI image and the PET-image. For example the multimodal image may have vector-valued image values, the vector at each image location in the multi-modal image having the image values of the APT-MRI data and of the PET-image data as its components. A multi-modal heterogeneity estimate corresponds to evaluation of a measure of heterogeneity of the values of this vector at locations in an image area. In yet another implementation a heterogeneity estimate in one of the APT-MRI data may be improved on the basis of image values, e.g. its heterogeneity estimate of the PET-image data (or vice-versa). For example, the heterogeneity estimate in the APT-MRI image may be weighted locally on the basis of the image values, e.g. its local heterogeneity estimate of the PET-image (or vice versa).

Combined use of amide proton transfer MRI image data and the PET image data with 18F-FLT, 11C-MET, or 18F-FDG PET as PET tracers (i.e. with the individual compounds or combinations thereof) provides for improved detection, localization and characterization of cell proliferation. Both amide proton transfer MRI imaging and the PET imaging primarily sense effects related to activity within cells. Amide proton transfer MRI imaging and the PET imaging can provide complementary information, because they detect activity in different metabolic pathways.

It is easy to provide an amide proton transfer MRI imaging system that captures images at higher spatial resolution than PET imaging. PET images with the described PET tracers are currently considered a gold standard for treatment assessment. When a combination of images from an amide proton transfer MRI imaging system and a PET imaging system are used where the amide proton transfer MRI imaging system provides information at higher spatial resolution than the PET imaging system, the PET image can be used to distinguish areas of interest and the amide proton transfer MRI image data can be used to enhance spatial resolution in combination with that distinction.

In an embodiment, data for image locations of the amide proton transfer MRI image are processed and/or displayed distinguished based on image values derived from the

PET image at the corresponding image locations. For example, the amide proton transfer MRI image may displayed with different coloring or contrast dependent on the corresponding PET image data, or selectively only where the PET image data meets a predetermined criterion, such as that the corresponding PET image data is within a predetermined range, e.g. above a threshold. As another example, when computing an image data measure from the amide proton transfer MRI image different image locations may be weighed differently dependent on the PET image data.

Tissue heterogeneity is an important factor for treatment assessment. It is known that heterogeneity measures computed from PET images for the described PET tracers can provide a useful estimate of tissue heterogeneity. The amide proton transfer MRI image may provide improved estimate of tissue heterogeneity because it has higher spatial resolution. In an embodiment a measure of multimodal heterogeneity is computed using the PET image and the amide proton transfer MRI image values as different modes in the multimodal heterogeneity computation. Inhomogeneity of an image of vector image values may be computed, wherein each image location has a vector value with a vector component dependent on the amide proton transfer MRI image value and a vector component derive from for the PET image representing joint occurrence of these values for a same location. As another example, contributions of different image regions of the amide proton transfer MRI image data to the measure of multimodal heterogeneity may be weighed dependent on the PET image data for those regions, for example dependent on PET image heterogeneity.

In an embodiment, classification of image areas is based on criteria that depend both on a value derived from the amide proton transfer MRI and a value derived from the PET image for the image locations and/or image areas. Thus a more elaborate classification is possible for treatment assessment. A classification may be defined for example by using the values derived from the amide proton transfer MRI and PET image as respective coordinates of a point in a virtual space, the classification depending on whether the point lies within a region in the virtual space that has been predefined for the class. In a further embodiment classification may involve classifying the amide proton transfer MRI data and PET data individually, for example dependent on whether they are in respective value ranges defined for the class, and assign combined classifications based on combinations of the individual classifications. Thus for example more classes can be used, in an image displaying local tissue classification, the classes corresponding to different combinations of individual classifications. As another example, a different class scope may be defined, such as a class containing only locations with predetermined individual classifications of the amide proton transfer MRI image and PET image. As another example a class scope may be defined of a class containing locations where at least one of the amide proton transfer MRI image and PET image has a predetermined individual classification.

In an embodiment, the PET image may be used to select a region of interest in the amide proton transfer MRI image for use in processing of the amide proton transfer MRI image. The image processing system may be configured to receive a user indication of an image location for example, and the image processing system may be configured to select a region of image locations containing the selected image location and further image locations that have similar PET image data as the selected image location, e.g. PET image data that does not differ more that a threshold amount from the PET image data at the selected image location, or where no image data edge is present between the selected location and the further location. In other embodiments the image processing system may select the region of interest without user input, for example by selecting image locations where the image data meets a predetermined criterion.

The computer program product may be used in an amide proton transfer MRI imaging system for example using a PET image as input, or in a workstation that has access to images obtained with amide proton transfer MRI imaging and PET imaging. In an embodiment, a combined PET-MRI scanner may be used.

The computer program product may be used in a PET-MRI imaging method, comprising
obtaining amide proton transfer MRI image data;
obtaining 18F-FLT, 11C-MET, or 18F-FDG PET image data;
making combined use of image values at corresponding image locations defined by the amide proton transfer MRI image data and the PET image data. For each amide proton transfer MRI scan a corresponding PET scan may be performed, but this may not be necessary. In an embodiment, amide proton transfer MRI scans of a subject are performed between successive treatment steps (e.g. radiation treatment and/or chemical treatment), and used in combination with 18F-FLT, 11C-MET or 18F-FDG PET data obtained at a single stage of treatment, for example before the successive treatment steps. Combined with amide proton transfer MRI scans from different stages, the PET image data obtained at a single stage may be sufficient for treatment assessment. In this way administration of PET tracers may be reduced.

### BRIEF DESCRIPTION OF THE DRAWINGS

These and other objects and advantageous aspects will become apparent from a description of exemplary embodiments, with reference to the following figures.

FIG. 1 shows a PET-MRI imaging system

FIG. 2 shows a PET-MRI imaging arrangement

### DETAILED DESCRIPTION OF EXEMPLARY EMBODIMENTS

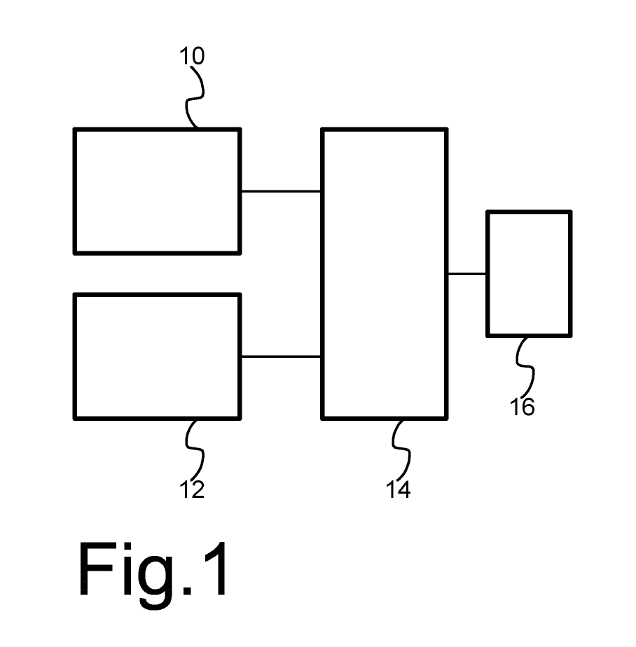

FIG. 1 shows a PET-MRI imaging system comprising PET-MRI imaging system for forming images of a subject 10. The PET-MRI imaging system comprises an MRI scanner 10, a PET scanner 12, an image processing system 14 and a display screen 16. Image processing system 14 is coupled to MRI scanner 10, PET scanner 12 and display screen 16. Image processing system 14 is configured to combine MRI and PET images from the MRI scanner and the PET scanner.

MRI scanner 10 is configured to perform amide proton transfer MRI of a subject. Amide proton transfer MRI is known per se. Amide proton transfer MRI comprises selectively saturating amide protons by irradiating a sample area of the subject with radio frequency (RF) electro-magnetic radiation This is followed by conventional MRI imaging of the sample area, for example MRI imaging of bulk water molecules, making use of the effect that proton exchange leads to a reduced amount of excitable water protons in the environment of the amide. Amide protons saturation requires relatively long RF irradiation, compared to Chemical Exchange-dependent Saturation Transfer (CEST) MRI using a special administered CEST contrast agent.

MRI scanner 10 may comprise a conventional MRI scanner subsystem. A conventional MRI scanner comprises one or more gradient magnets configured to produce a magnetic field in a sample area, an RF generator, an RF receiver, RF transmission antennas coupled to the RF generator and RF receiver and configured to generate and receive RF fields from the sample area, and a signal processing system. The latter may be part of image processing system 14. To perform amide proton transfer MRI, MRI scanner 10 may comprise control software configured to
make MRI scanner 10 generate an RF signal at a resonance frequency of amide protons,
cause MRI scanner 10 to transmit the RF signal at that resonance frequency using a combination of RF power and duration that is sufficient to cause saturation in the sample area and subsequently to
make MRI scanner 10 perform conventional MRI imaging to determine (water) proton response to RF fields as a function of position in the sample region.

Both the resonance frequency of protons in water and that of amide protons depend on the magnetic field, but at any given magnetic field the resonance frequency of the amide protons lies shifted with respect to that of the water protons. The amount of shift as a function of the magnetic field is known per se so that the amide proton resonance frequency can be determined in advance, but optionally the required resonance frequency may be determined dynamically, for example by measuring water proton responses after saturating irradiation at different frequencies in a range that includes the amide proton resonance frequency, and selecting the RF frequency for saturation based on those responses. The frequency may be set at a frequency for which the responses at different frequencies indicate maximum response.

The saturating irradiation may be applied as an RF pulse or pulse sequence with a duration of between one to ten seconds for example and an RF magnetic field amplitude of between one to ten micro Tesla for example.

In a preferred embodiment differential amide proton imaging is used. This comprises a first MRI imaging operation after saturation by RF irradiation at the resonance frequency Fa of the amide protons and a second MRI imaging operation after saturation by RF irradiation tuned to a frequency Fb =2*Fw−Fa, where Fw is the resonance frequency of protons of water. In this embodiment, the APT-MRI image is formed by subtracting image values of images formed by means of the first and second MRI imaging operations. Thus contributions of non-amide protons are removed or at least reduced. The first and second MRI imaging operations may each be performed with the same time delay after their corresponding saturation. Alternatively, a difference between the MRI images obtained with and without saturation at the amide proton resonance frequency may be used.

The MRI image difference obtained by means of saturation may be normalized by dividing its image values by image values obtained without preceding saturation.

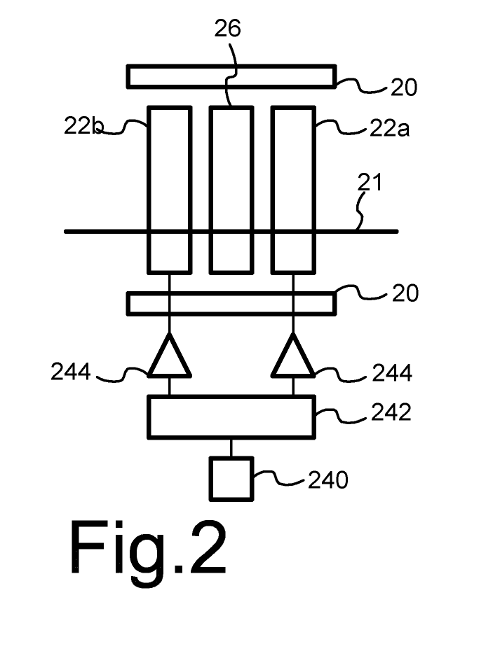

FIG. 2 schematically shows a PET-MRI imaging tunnel arrangement. A subject carrier surface 21 is symbolically shown. The arrangement comprises a magnet coil 20, a first and second ring shaped RF saturation coil 22a,b and a ring shaped gamma ray detector array 26 coaxially located between first and second RF saturation coils 22a,b. Furthermore an RF saturation signal generator 240, a multiplexer 242, and first and second amplifiers 244 coupled between multiplexer 242 and RF saturation coils 22a,b are shown. Multiplexer 242 is used to switch between supplying RF saturation to saturation coils 22a,b via first and second amplifier respectively during application of RF radiation for saturation. A method of operating such an APT MRI imaging system is described in co-pending European patent application no 13166255.3, which does not include a PET detector. The coils are shown schematically. Although the coils may be wound in a ring shape, other forms may be used, as described in WO2011086512.

In an embodiment, the MRI scanner is equipped with a multi-mode or multi-element volume transmit coil for RF irradiation, and multiple RF power amplifiers that are enabled repetitively and in an alternating fashion to improve efficiency of the RF power amplifier performance. In the integrated PET-MRI system, such multi-element volume transmit coils are particular useful as they create a gap as free path from the region of interest of the subject under investigation to the PET detectors.

MRI scanner 10 may comprise an amplifier or amplifier system for amplifying the tuned RF signal.

As an alternative contrast generating mechanism, electrical properties tomography, or EPT MRI can be applied independent or integrated with the APT MRI acquisition. This method applies signal processing of the phase of the MR image to derive the local conductivity (in Siemens/meter) of the region of interest.

PET scanner 12 may be implemented as a conventional PET scanner. A conventional PET scanner comprises a gamma ray detector system and a signal processing system. Part or all of the latter may be part of image processing system 14.

PET scanner 12 is configured to determine gamma ray emission intensity as a function of position in the sample region. In PET scanning gamma ray pairs resulting from positron-electron annihilation involving positrons emitted from a PET tracer are detected. A

PET scanner may comprise an array of gamma ray detectors in a ring around a subject, and a signal processing circuit coupled to the gamma ray detectors, the signal processing circuit being configured to detect substantially coincident gamma ray detections from different detectors (substantially meaning temporally not more distant than explicable by differences in travel distance a position in the scanner to different detectors). Furthermore the signal processing circuit of the PET scanner is configured to determine location information from the locations of the detectors that detected the gamma rays in the pair and/or detection timing.

Prior to PET scanning a PET tracer is administered to the subject. This may be done by oral ingestion for example, or intravenously. In an embodiment ¹⁸F-desoxyglucose (¹⁸F-FLT) may be used as PET tracer. This tracer is known for brain tumor PET studies.

Oncologic treatment planning and assessment of cancer treatment response needs to differentiate benign and malignant tissues at high sensitivity and specificity. Differentiation may be based on detection of cell proliferation. Indications of cell proliferation may be obtained using imaging biomarkers, such as 18 F FLT (fluoro-3′-deoxy-3′L-fluorothymidine). Upregulated DNA synthesis requires increased amounts of thymidine in proliferating cells, and 18F FLT used in the thymidine salvage pathway for DNA synthesis is trapped upon phosphorylation by TK1 in certain cell types. The location of cells where this occurs can be detected b PET imaging. In addition, 18F FLT PET imaging shows intra-tumour heterogeneity of proliferation in certain tumours to correlate with predicted chemotherapeutic response (J Nucl Med 2012; 53 (Supplement 1):387).

It may be noted that 18F FLT is not trapped in all cell types. See e.g. E. T. McKinley et al. Limits of [18F]-FLT PET as a Biomarker of Proliferation in Oncology (PLOS ONE Vol 8, Issue 3, e58938).

18F FLT PET results in information that is complementary to the information that is provided by APT MRI.

It is known that, among others, the APT MRI image is also indicative of processes within cells. This may be contrasted with conventional MRI techniques that detect mostly protons of water outside cells, e.g. in the form of tumor associated edema or necrosis. In contrast, APT MRI image shows locations of protein production associated with chromosome reproduction and hence cell replication activity.

The PET image obtained with ¹⁸F-FLT is indicative of ribosome activity. It is known that ¹⁸F-FLT PET images are also indicative of locations of cell replication activity. In other embodiments, 11C-MET, or 18F-FDG may be used, or combinations of two or more of 18F-FLT, 11C-MET, or 18F-FDG. Each of these is known per se as a PET tracer. In the following 11C-MET, 18F-FDG, or combinations thereof or with 18F-FLT may be substituted for 18F-FLT. The images obtained by MRI and PET may be three dimensional images. In an alternative embodiment two dimensional images may be used, e.g. of slices or projections.

For PET images it is known to use image processing to evaluate heterogeneity of a tumor or part of a tumor. Heterogeneity characterizes aspects of variation of the PET image values within the tumor or part of the tumor. A number of measures of inhomogeneity of image values of a PET image or image part that shows a tumor or part of a tumor are known per se. For response assessment, such inhomogeneity metrics comprise a number of non-spatially resolved measures like Coefficient of Variation (CV), skewness, kurtosis, or entropy of the signal intensity distribution; as well as localized measures like the grey-level co-occurrence matrix and its constituents e.g. dissimilarity and homogeneity. The application of such measures is described in an article by Willaime et al, titled “Quantification of intra-tumour cell proliferation heterogeneity using imaging descriptors of 18F fluorothymidine-positron emission tomography”, published in Phys. Med. Biol. 58 (2013) 187-203. Willaime et al list and evaluate a set of alternative descriptors for use as a measure of heterogeneity in texture analysis of a PET image (see Williame et al table 2, incorporated by way of reference herein).

To make combined use of the amide proton transfer MRI image data and 18F-FLT, 11C-MET, or 18F-FDG PET image data, amide proton transfer MRI and 18F-FLT, 11C-MET, or 18F-FDG PET images may be registered, i.e. a map may be determined that map locations in the image spaces onto each other that represent the same location in a subject.

MRI scanner 10 has a higher spatial resolution than PET scanner 12. Generally it is easier to provide higher resolution with MRI than with PET, due to the higher signal to noise ratio of the MRI imaging data. This is also the case for APT MRI. This makes it possible to realize a more reliable evaluation of tumor heterogeneity using the increased spatial resolution of the APT MRI image, while the PET heterogeneity measures are generally compromised by partial volume effects. In an embodiment, image processing system 14 may be configured to upsample the PET image so as to make the number of image locations (pixels or voxels) of the PET and APT MRI images equal, but in this case the APT MRI image has a wider spatial frequency bandwidth of physically relevant content than that of the PET image.

When the amide proton transfer MRI and PET images have different sampling grids of locations in the subject, one or more of the images may be resampled by means of interpolation to enable a pixel to pixel or voxel to voxel registration. Alternatively, the registration may merely define the location of pixels/voxels in one image relative to those of the other image, enabling the determination of an image value for a corresponding location in the other image by interpolation, or taking the image value of a pixel/voxel that is mapped to a region that includes the corresponding location in the other image etc. Information derived from image value for such corresponding locations in any such way will be termed mutually registered image information.

Image processing system 14 may be configured to combine mutually registered image information from the APT MRI and PET images in one or more of a number of ways.

In a first embodiment, image processing system 14 may be configured to use the PET image as a selector for selecting image locations in the APT MRI image. Selection may be part of evaluation of the APT MRI image for example, image processing system 14 using the PET image based selection to select image locations from the APT-MRI image that image processing system 14 will use for the evaluation. Selection may take the form of control of display of the APT MRI image by image processing system 14, in a way that distinguishes image locations dependent on PET image values. For example, image processing system 14 may display a APT MRI image value for an image location differently (or not at all) dependent on whether the PET image value for the image location is in a predetermined range or not.

In a second embodiment, image processing system 14 may be configured to use the PET image and the APT MRI image together to select image regions. When respective predetermined image value ranges are defined for the PET image and the APT MRI image individually, four classes of image locations may be distinguished, dependent on whether or not the PET image and the APT MRI image are in the predetermined ranges. Derived types of classes include a class wherein both the image values of an image location are in the corresponding range and a class wherein at least one of the image values of an image location is in the corresponding range. Other types of classes include classes defined for respective areas in a two dimensional plot with points that have plot coordinates derived for the PET and APT MRI image respectively. In this case, the class may be assigned dependent on PET and APT MRI image data according to the location in the plot. The four mentioned classes obtained with individual image value ranges for the PET image and the APT MRI image correspond to rectangular areas in such a plot, but other classes may be defined that correspond to areas of other shapes.

Image processing system 14 may be configured to generate an image that indicates the class of each image location. As in the case of the first embodiment, the select image regions may be used as part of part of evaluation of the APT MRI image and/or control of display of the APT MRI image. Image processing system 14 may be configured to perform combined FLT and APT classification using predefined threshold values for high/low PET FLT and high/low APT-MRI image values. In an exemplary embodiment, these thresholds are a mean Standardized Uptake Value (SUV) of 2.3 for FLT PET, and an APT MRI signal change of 3% of the water signal without saturation transfer at a magnetic field of 3T with saturation during 2 seconds. However, other thresholds may be used. Image processing system 14 may provide for user controlled setting of the thresholds.

In a third embodiment, image processing system 14 may be configured to evaluate multimodal heterogeneity, i.e. inhomogeneity of a multimodal PET+APT MRI image. For each image location, the PET and APT MRI images may be considered to provide a vector of image data, wherein the vector components are PET and APT MRI image values for that location respectively, or combinations thereof. Evaluation of multimodal heterogeneity corresponds to evaluation of a measure of heterogeneity of the values of this vector at locations in an image area.

In an embodiment, the image processing system 14 applies histogram analysis methods as described by Willaime et al to APT MRI data as well as to EPT MRI data to compute a measure of tumour heterogeneity for quantification and evaluation of tumour heterogeneity.

In another embodiment, predetermined value ranges are defined, for example in terms of a lower threshold, or a lower and upper threshold, and image processing system 14 is configured to compute a volume from a count of image locations within the predetermined range. Alternatively image processing system 14 may compute cumulative intensity-volume histograms, with counts of image locations with an image value equal to or higher than a lower threshold value, as a function of the lower threshold value.

In another embodiment, image processing system 14 is configured to compute a ratio of the cumulative intensity volumes for two selected contrast mechanisms, like APT and EPT, or APT and diffusion MRI ADC values, or EPT and regional perfusion blood volume, and the like.

In another embodiment, the image processing system 14 co-registers PET and MR images, selects a region of interest and computes a heterogeneity measure of the APT or EPT MRI image selectively in the selected region of interest. Image processing system 14 may configured to select the region of interest based on user interaction and/or based on threshold values for PET image values.

In another embodiment, image processing system 14 is configured to apply cluster analysis to selectively generate and display an overlay image generated from e.g. 18F FLT signal intensities in a predefined range of values, with e.g. APT images of a predefined set of enhancement percentages. Four images may be generated with high FLT, high APT; low FLT, high APT; low FLT, low APT; and high FLT, low APT. Cluster analysis may comprise a determination whether image values are in a predetermined range. But alternatively other known cluster analysis techniques may be used, such as techniques based on histograms, region growing etc.

When the APT MRI and PET images show that the APT MRI and PET images of the tumor are correlated, this indicates that the APT MRI image is suitable for computation of the homogeneity and monitoring treatment response. This can be used to avoid overbroad detection by APT MRI. Image processing system 14 computes a measure of homogeneity of image values of the APT MRI image or part thereof from MRI scanner 10. Because the measure of homogeneity of image is based on an APT MRI image from MRI scanner 10 with higher spatial resolution than the PET image from PET scanner 12, a more reliable value of the measure of homogeneity is made possible, which makes more different measures of homogeneity suitable for treatment assessment than in the case of PET images alone.

In an embodiment, image processing system 14 may be configured to compute the measure of homogeneity for a plurality of blocks of APT-MRI image locations, and image processing system 14 may be configured to form and display a heterogeneity image representing values of the measure of homogeneity at different locations.

In an embodiment, image processing system 14 may be configured to use location dependent data, such as intensity, derived from the PET image to modulate display of the heterogeneity image from the APT-MRI image as a function of location in the image. For example, in an embodiment, image processing system 14 may be configured to compare the location dependent data derived from the PET image with a threshold value and to perform modulation by enabling or disabling display of the heterogeneity image at image locations corresponding to the locations associated with the data from the PET image, dependent on the comparison. Other forms of modulation may include more graded amplitude modulation dependent on the data from the PET image.

In an embodiment, image processing system 14 may be configured to compute a further measure of homogeneity from the PET image, e.g. a plurality of such values for respective blocks, and image processing system 14 may be configured to display a combined image with image values based on combinations of the values of the measure heterogeneity for the PET and APT-MRI images. Image processing system 14 may be configured to upscale the resolution of the image with values of the measure of heterogeneity obtained from the PET image, for example by interpolation, to the resolution of the image with values of the measure of heterogeneity obtained from the APT-MRI image and combine the values for corresponding locations in these images to form the combined image.

In an embodiment image processing system 14 is configured to compute values of measures of heterogeneity for corresponding regions from the APT MRI image and the PET image. In embodiment image processing system 14 is configured to generate and display a plot of the values of the measures of heterogeneity of the PET image values versus those of the APT-MRI image values. Image processing system 14 may be configured to display further plots wherein values of the measure of heterogeneity of the PET image values or the APT-MRI image values are plotted versus values of a measure of heterogeneity of MRI contrast like diffusion ADC, FA, kurtosis, spectroscopy choline levels

When and/or where the APT-MRI image exhibits correlation with the ¹⁸F-FLT PET image, this ensures that the tumor is of a type that can be detected by APT-MRI. In this case, assessment can be performed using the APT-MRI images, without the burden of additional ¹⁸F-FLT PET scans after respective treatment steps. In an embodiment, image processing system 14 is configured to apply heterogeneity computations to the MRI images from MRI scanner 10.

In an embodiment ¹⁸F-FLT PET is used to form a PET image at a first stage prior to a treatment step and APT MRI is used to form an APT-MRI at this stage prior to the treatment step and a further APT-MRI image after that treatment step, or a plurality of APT-MRI images each after a respective treatment step in a succession of treatment steps. Herein the treatment steps may be radiotherapy and/or chemotherapy steps. The first stage may be a stage prior to all treatment steps of a therapy, or it may be an intermediate stage between successive treatment steps.

In an embodiment, image processing system 14 is configured to derive values of the texture analysis metrics for APT MRI, or combined metrics based on PET image intensity based MRI analysis pre- and post-treatment and to plot as these values in a parametric response map. In such a map, the per-image location pre-treatment values (from image locations that may be voxels or pixels) are plotted on the x-axis and the per-image location post-treatment values on the y-axis. Values that are far from the orthogonal axis indicate image locations with large changes of its value. Based on this change criterion for one of the contrasts, like APT MRI or EPT MRI, image processing system 14 may perform texture analysis or volume fraction analysis for the other contrasts, e.g. the 18F FLT values, at the image locations with changed parameter values, and at the image locations that do not show signal changes. Such analysis selectively shows cell proliferation from e.g. 18F FLT in the voxels that respond or do not respond.

When the APT-MRI image obtained at the first stage exhibits correlation with the ¹⁸F-FLT PET image, this ensures that the tumor is of a type that can be detected by APT-MRI. In this case, assessment can be performed using the APT-MRI images, without the burden of additional ¹⁸F-FLT PET scans after respective treatment steps.

In an embodiment PET scanner 12 may be separate from MRI scanner 10. In this embodiment PET scanner 12 may be configured to transmit ¹⁸F-FLT PET image data to image processing system 14 for use in processing the APT MRI images.

In another embodiment, a combined APT MRI/PET system is used, comprising MRI/PET scanners that are both capable of performing measurements while the subject is in the same position in a scanner. Designs of combined PET-MRI scanners that enable such measurements are known per se. More preferably APT MRI/PET system is used, that is capable of performing PET and MRI measurements substantially simultaneously. This facilitates registration of the PET and MRI images. Alternatively, an MRI/PET system is used PET and MRI scans of a subject are performed when the subject is at respective different positions. In this case, image processing system 14 may be configured to register the PET and MRI images prior to their combination.

In an embodiment, image processing system 14 is configured to combine registered MRI and PET images from MRI scanner 10 and PET scanner 12. Any one of a range of combination methods may be used. For example, image processing system 14 may be configured to detect locations in the APT-MRI and/or PET images of the sample region at which the APT-MRI and PET signals exceed respective threshold values and form a combine image with image values that that single out locations where this is the case. Image processing system 14 may cause the combined image to be displayed on display screen 16. In a further embodiment image processing system 14 may use the combined image to evaluate properties within image areas by weighing measured properties for image location according to whether the APT-MRI and/or PET signals exceed respective values. In other embodiments the APT-MRI and PET signals may be combined in other ways, for example by controlling the luminance channel of each image location of the combined image dependent on the APT-MRI signal for that image location and controlling the color saturation channel dependent on the PET signal for that image location, or any other way of combining the signals at the image location. In another embodiment, image processing system 14 may be configured to compute a difference image between the PET image and the APT-MRI image and display an image representing this difference.

Instead of combining individual APT-MRI images with a PET image, difference images between APT-MRI images may be combined with the PET image. Although the application of image processing operations for evaluating heterogeneity to APT-MRI has been shown in combination with use of PET image information, it should be appreciated that application of such image processing operations to APT-MRI may also yield useful information when not combined with use of PET image information.

According to another aspect, EPT MRI imaging may be used instead of the described use of APT-MRI or in combination APT-MRI to form combined APT-EPT MRI images for use instead of the described use of APT-MRI.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer program product with instructions for a programmable image processing system that, when executed by the programmable image processing system, will make the programmable image processing system perform the steps of
obtaining amide proton transfer MRI image data;
obtaining 18F-FLT, 11C-MET, or 18F-FDG PET image data;
making combined use of image values at corresponding image locations defined by the amide proton transfer MRI image data and the PET image data.

2. A computer program product according to claim 1, wherein the instructions are configured to make the programmable image processing system make said combined use of the image values by evaluating a measure of multimodal heterogeneity of jointly occurring amide proton transfer MRI and PET image values.

3. A computer program product according to claim 1, wherein the instructions are configured to make the programmable image processing system make said combined use of the image values in image processing and/or image display of the amide proton image, wherein data for image locations of the amide proton image are processed and/or displayed distinguished based on image values derived from the PET image at the corresponding image locations.

4. A computer program product according to claim 3, wherein the instructions are configured to make the programmable image processing system assign combined classifications to image locations and/or image areas in the amide proton transfer MRI and/or PET image data based on joint occurrence of values derived from the amide proton transfer MRI and PET image for the image locations and/or image areas.

5. A computer program product according to claim 4, wherein the instructions are configured to make the programmable image processing system
assign first classifications to the image locations and/or image areas based on the amide proton transfer MRI data for the data image locations and/or image areas;
assign second classifications to image locations and/or image areas based on the PET image data for the data image locations and/or image areas;
assign a further classification based on a combination of the first and second classifications.

6. A computer program product according to claim 3, wherein the instructions are configured to make the programmable image processing system
select a region of interest image based on the values of the PET image;
compute a heterogeneity measure of the APT MRI image selectively in the selected region of interest.

7. A computer program product according to claim 3, wherein the instructions are configured to make the programmable image processing system select the region of interest based on a received user indication on a display screen showing the PET image and/or based on threshold values for PET image values.

8. A PET-MRI imaging system comprising an image processing system programmed with a computer program product according to claim 1.

9. A PET-MRI imaging system according to claim 8, comprising an amide proton transfer MRI imaging system coupled to the image processing system.

10. A PET-MRI imaging system according to claim 8, comprising a PET imaging system coupled to the image processing system for providing the 18F-FLT, 11C-MET, or 18F-FDG PET image data.

11. A PET-MRI imaging method, comprising
obtaining amide proton transfer MRI image data;
obtaining 18F-FLT, 11C-MET, or 18F-FDG PET image data;
making combined use of image values at corresponding image locations defined by the amide proton transfer MRI image data and the PET image data.

12. A PET-MRI imaging method according to claim 11, comprising
performing respective amide proton transfer MRI scans of a subject at stages between respective steps of a therapy of the subject;
combining images derived from the respective amide proton transfer MRI scans each with said 18F-FLT, 11C-MET or 18F-FDG PET image.

13. A PET-MRI imaging method according to claim 11, comprising
administering a PET tracer selected from the group of 18F-FLT, 11C-MET and 18F-FDG and combinations thereof to a subject;
performing a PET scan of a sample region in the subject to form the PET image.
